(19) United States
(12) Patent Application Publication
(10) Pub. No.: US 2025/0195843 A1
(43) Pub. Date: Jun. 19, 2025

(54) Bulge Anchor for Urinary Catheter

(71) Applicant: C. R. Bard, Inc., Franklin Lakes, NJ (US)

(72) Inventors: Vijaya Pratap V N, Thanjavur (IN); Vivek Raju, Ernakulam (IN)

(21) Appl. No.: 18/840,824

(22) PCT Filed: Feb. 24, 2022
(86) PCT No.: PCT/US22/17734

§ 371 Date: Aug. 22, 2024

(51) Int. Cl. A61M 25/04; A61M 25/00; A61M 39/24

(52) U.S. Cl. CPC A61M 25/04; A61M 25/0017; A61M 2025/0004; A61M 2039/2426

(57) ABSTRACT

Embodiments disclosed herein are directed to a urinary catheter having a bulge anchor configured to transition from an insert mode, where the catheter can advance through the urethra, to an anchor mode preventing the catheter from being withdrawn proximally. Often a clinician is required to estimate when the anchor is within the bladder to prevent premature deployment of the anchor, causing trauma to the patient. Embodiments, include an indicator aperture configured to provide a first fluid flow to indicate when the anchor is correctly positioned. The clinician can the actuate a locking handle to deploy the anchor and place a drainage aperture in fluid communication with the bladder to provide a second fluid flow, greater than the first fluid flow.

### SUMMARY

Briefly summarized, embodiments disclosed herein are directed to bulge anchors for urinary catheters. Balloon anchored urinary catheters, e.g. Foley catheters, etc., rely on expanding a distal portion of the catheter within the bladder to anchor the distal tip therein. A clinician must estimate a length of the urethra to determine when the balloon anchor is within the bladder before expanding to the anchored position. If the balloon anchor is deployed prematurely the balloon can expand within the neck of the bladder or the urethra, causing trauma to the patient. Similarly, over inserting the catheter can cause the tip of the catheter to impinge on a wall of the bladder causing pain or discomfort. In addition, balloon catheters include a drainage eyelet disposed distally of an anchoring balloon. As such, when the catheter is anchored within the bladder, a portion of fluid can collect between the drainage eyelet and the neck of the bladder leading to stagnation of fluid with the bladder. Embodiments disclosed herein are directed to the foregoing.

Disclosed herein is an indwelling urinary catheter including, a catheter body defining a lumen, and an anchor disposed at a distal end of the catheter body and transitionable between an insert mode and an anchor mode, the anchor including, a first slit valve and a second slit valve each extending longitudinally and defining a first arm and a second arm, an indicator aperture disposed proximally of one or both of the first slit valve and the second slit valve, and configured to provide a first fluid flow to the lumen in both the insert mode and the anchor mode, and wherein the first slit valve and the second slit valve are each configured to define a drainage aperture in the anchor mode to allow a second fluid flow into the lumen and to prevent the second fluid flow into the lumen in the insert mode.

In some embodiments, the first fluid flow is less than the second fluid flow.

In some embodiments, the indicator aperture communicates with one of the first slit valve or the second slit valve.

In some embodiments, one or both of the indicator aperture and the drainage aperture extends through a side wall of the catheter body to communicate with the lumen.

In some embodiments, the first arm and the second arm define a first outer diameter in the insert mode and a second outer diameter in the anchor mode, the first diameter being less than the second diameter.

In some embodiments, the indwelling urinary catheter further includes a locking handle coupled to a proximal end of the catheter body, and a string extending through the lumen, a proximal end of the string is coupled to a female piece of the locking handle, and a distal end of the string is coupled to a distal tip of the catheter body, a proximal end of the catheter body is coupled a male piece of the locking handle, the female piece slidably engaged with the male piece to transition the anchor between the insert mode and the anchor mode.

In some embodiments, the female piece is configured to slide proximally relative to the male piece to transition the anchor to the anchor mode.

In some embodiments, the female piece includes a channel having a distal lateral channel communicating with a longitudinal channel which communicates with a proximal lateral channel, the male piece includes a post extending from an outer surface thereof, the post slidably engaged with the distal lateral channel in the insert mode, the proximal lateral channel in the anchor mode, and the longitudinal channel when transitioning therebetween.

Also disclosed is a urinary catheter including, an outer catheter including a distal opening, and a first handle coupled to a proximal end thereof, and an inner catheter arranged co-axially and slidably engaged with the outer catheter, the inner catheter including, an anchor disposed distally and transitionable between an insert mode and an anchor mode, an indicator aperture disposed at a distal tip of the inner catheter, a drainage aperture disposed proximally of the anchor, and a second handle coupled to a proximal end thereof.

In some embodiments, the anchor is configured to be advanced distally of the distal opening to transition to the anchor mode, and withdrawn proximally of the distal opening to transition to the insert mode.

In some embodiments, the first handle is co-axially arranged and slidably engaged with the second handle along a longitudinal axis between a first configuration and a second configuration to transition the anchor between the insert mode and the anchor mode.

In some embodiments, the first handle includes a post extending from an outer surface thereof and the second handle includes a channel having a distal lateral channel communicating with a longitudinal channel which communicates with a proximal lateral channel, the post slidably engaged with the proximal lateral channel in the insert mode, slidably engaged with the distal lateral channel in the anchor mode, and slidably engaged with the longitudinal channel when transitioning between the insert mode and anchor mode.

In some embodiments, the anchor is biased towards one of a J-shape, or a pigtail shape, in the anchor mode and elastically deformed to a linear shape in the insert mode when the anchor is withdrawn proximally into a distal portion of the outer catheter.

In some embodiments, the anchor is biased towards a linear shape and further includes a tether extending between a distal tip of the inner catheter and a distal tip of the outer catheter, the tether configured to elastically deform the anchor to a non-linear configuration when the anchor is advanced distally of the distal opening.

In some embodiments, the anchor is biased towards a linear shape and further includes first magnetic element disposed in a distal tip of the inner catheter and a second magnetic element disposed in a wall of the inner catheter, proximally of the anchor, the first magnetic element configured to be attracted to the second magnetic element when the second magnetic element is advanced distally of the distal opening and elastically deform the anchor to a non-linear configuration.

Also disclosed is an indwelling urinary catheter including, a catheter body defining a lumen, an anchor structure disposed at a distal end of the catheter body and including a tether integrally formed therewith extending from a distal tip of the catheter body to a proximal end of the anchor structure, an aperture extending through a wall of the catheter body and communicating with the lumen, the aperture disposed proximally of the anchor structure, and a push rod extending through the lumen and configured to be urged distally to elastically deform the anchor structure from an insert mode defining a first diameter, to an anchor mode defining a second diameter, larger than the first diameter.

In some embodiments, the aperture in the insert mode defines a first aperture diameter to provide a first fluid flow, and wherein the aperture in the anchor mode defines a second aperture diameter, larger than the first aperture diameter to provide a second fluid flow, greater than the first fluid flow.

Also disclosed is a method of draining a fluid from a bladder of a patient including, advancing a distal tip of a catheter through a urethra into the bladder, the catheter including an anchor, an indicator aperture, and a drainage aperture, advancing the indicator aperture into the bladder to provide a first fluid flow and indicating the anchor is correctly positioned for transitioning from an insert mode to an anchor mode, transitioning a locking handle between a first configuration and a second configuration, transitioning the anchor from the insert mode to the anchor mode, and providing fluid communication between the drainage aperture and the bladder to provide a second fluid flow, different from the first fluid flow.

In some embodiments, the drainage aperture includes a slit valve disposed proximally of the indicator aperture and transitions from a closed position in the insert mode which prevents the second fluid flow, to an open position in the anchor mode which provides the second fluid flow.

In some embodiments, the catheter includes an outer catheter body and an inner catheter body, slidably engaged and co-axially aligned therewith, and wherein transitioning the anchor to the anchor mode includes advancing the anchor and the drainage aperture distally of a distal tip of the outer catheter body.

In some embodiments, the method further includes a tether extending between a distal tip of the inner catheter body and a distal tip of the outer catheter body and elastically deforming a distal portion of the inner catheter body to a non-linear configuration.

In some embodiments, the method further includes a first magnetic element disposed in a tip of the inner catheter body, and a second magnetic element disposed in a wall of the inner catheter body proximally of the anchor, and wherein transitioning the anchor to the anchor mode includes advancing the first magnetic element and the second magnetic element distally of the distal tip of the outer catheter body to allow the first magnetic element to couple with the second magnetic element elastically deforming a distal portion of the inner catheter body to a non-linear configuration.

In some embodiments, transitioning the anchor to an anchor mode further includes advancing a push rod distally through a lumen of the catheter to elastically deform the anchor, the anchor including a tether formed integrally therewith and extending from a distal tip of the catheter to a proximal end of the anchor.

Also disclosed is an indwelling urinary catheter including, a catheter body defining a lumen, a locking handle coupled to the proximal end of the catheter body, and an anchor disposed at a distal end of the catheter body and transitionable between an insert mode and an anchor mode, the anchor including, an indicator aperture disposed at a first longitudinal position, a drainage aperture disposed at a second longitudinal position, proximal of the first longitudinal position, and a string extending from a distal tip of the catheter body over an outer surface of the anchor, through the indicator aperture and through the lumen of the catheter body, a proximal end of the string coupled to the handle.

### DRAWINGS

A more particular description of the present disclosure will be rendered by reference to specific embodiments thereof that are illustrated in the appended drawings. It is appreciated that these drawings depict only typical embodiments of the invention and are therefore not to be considered limiting of its scope. Example embodiments of the invention will be described and explained with additional specificity and detail through the use of the accompanying drawings in which:

FIG. 1 shows an exemplary urinary catheter including a bulge anchor and a locking handle, in accordance with embodiments disclosed herein.

FIG. 2A shows a locking handle in a first configuration, in accordance with embodiments disclosed herein.

FIG. 2B shows a locking handle in a second configuration, in accordance with embodiments disclosed herein.

FIG. 2C shows an exploded view of a locking handle, in accordance with embodiments disclosed herein.

FIGS. 2D-2E show close up detail of the locking handle of FIG. 2C, in accordance with embodiments disclosed herein.

FIG. 3 shows a distal end of the catheter of FIG. 1 in an anchor mode and disposed within a bladder, in accordance with embodiments disclosed herein.

FIG. 4A shows a distal end of the catheter of FIG. 1 in an insert mode, in accordance with embodiments disclosed herein.

FIG. 4B shows a distal end of the catheter of FIG. 1 in an anchor mode, in accordance with embodiments disclosed herein.

FIG. 5A shows a perspective view of a catheter including a loop anchor, in accordance with embodiments disclosed herein.

FIG. 5B shows a schematic view of the catheter of FIG. 5A, in accordance with embodiments disclosed herein.

FIG. 5C shows a distal end of the catheter of FIG. 5A in an insert mode, in accordance with embodiments disclosed herein.

FIG. 5D shows a distal end of the catheter of FIG. 5A in an anchor mode, in accordance with embodiments disclosed herein.

FIG. 5E shows a distal end of the catheter of FIG. 5A in an anchor mode disposed within a bladder, in accordance with embodiments disclosed herein.

FIG. 6A shows a perspective view of a catheter including a locking handle and a J-anchor, in accordance with embodiments disclosed herein.

FIG. 6B shows a schematic view of the catheter of FIG. 6A, in accordance with embodiments disclosed herein.

FIG. 6C shows a distal end of the catheter of FIG. 6A in an insert mode, in accordance with embodiments disclosed herein.

FIG. 6D shows a distal end of the catheter of FIG. 6A in an anchor mode, in accordance with embodiments disclosed herein.

FIG. 6E shows an exploded view of the locking handle of FIG. 6A, in accordance with embodiments disclosed herein.

FIG. 6F shows close up detail of the locking handle of FIG. 6E, in accordance with embodiments disclosed herein.

FIG. 7A shows a perspective view of a catheter including a tethered J-anchor, in accordance with embodiments disclosed herein.

FIG. 7B shows a schematic view of the catheter of FIG. 7A, in accordance with embodiments disclosed herein.

FIG. 8 shows a schematic view of a catheter including a magnetic J-anchor, in accordance with embodiments disclosed herein.

FIG. 9A shows a perspective view of a catheter including a push-rod anchor, in accordance with embodiments disclosed herein.

FIG. 9B shows a schematic view of the catheter of FIG. 9A, in accordance with embodiments disclosed herein.

FIG. 9C shows a distal end of the catheter of FIG. 9A in an insert mode, in accordance with embodiments disclosed herein.

FIG. 9D shows a distal end of the catheter of FIG. 9A in an anchor mode, in accordance with embodiments disclosed herein.

FIG. 9E shows a distal end of the catheter of FIG. 9A in an anchor mode disposed within a bladder, in accordance with embodiments disclosed herein.

### DESCRIPTION

Before some particular embodiments are disclosed in greater detail, it should be understood that the particular embodiments disclosed herein do not limit the scope of the concepts provided herein. It should also be understood that a particular embodiment disclosed herein can have features that can be readily separated from the particular embodiment and optionally combined with or substituted for features of any of a number of other embodiments disclosed herein.

Regarding terms used herein, it should also be understood the terms are for the purpose of describing some particular embodiments, and the terms do not limit the scope of the concepts provided herein. Ordinal numbers (e.g., first, second, third, etc.) are generally used to distinguish or identify different features or steps in a group of features or steps, and do not supply a serial or numerical limitation. For example, “first,” “second,” and “third” features or steps need not necessarily appear in that order, and the particular embodiments including such features or steps need not necessarily be limited to the three features or steps. Labels such as “left,” “right,” “top,” “bottom,” “front,” “back,” and the like are used for convenience and are not intended to imply, for example, any particular fixed location, orientation, or direction. Instead, such labels are used to reflect, for example, relative location, orientation, or directions. Singular forms of “a,” “an,” and “the” include plural references unless the context clearly dictates otherwise.

In the following description, the terms “or” and “and/or” as used herein are to be interpreted as inclusive or meaning any one or any combination. As an example, “A, B or C” or “A, B and/or C” mean “any of the following, A, B, C, A and B, A and C, B and C, A, B and C.” An exception to this definition will occur only when a combination of elements, components, functions, steps or acts are in some way inherently mutually exclusive.

With respect to “proximal,” a “proximal portion” or a “proximal end portion” of, for example, a catheter disclosed herein includes a portion of the catheter intended to be near a clinician when the catheter is used on a patient. Likewise, a “proximal length” of, for example, the catheter includes a length of the catheter intended to be near the clinician when the catheter is used on the patient. A “proximal end” of, for example, the catheter includes an end of the catheter intended to be near the clinician when the catheter is used on the patient. The proximal portion, the proximal end portion, or the proximal length of the catheter can include the proximal end of the catheter; however, the proximal portion, the proximal end portion, or the proximal length of the catheter need not include the proximal end of the catheter. That is, unless context suggests otherwise, the proximal portion, the proximal end portion, or the proximal length of the catheter is not a terminal portion or terminal length of the catheter.

With respect to “distal,” a “distal portion” or a “distal end portion” of, for example, a catheter disclosed herein includes a portion of the catheter intended to be near or in a patient when the catheter is used on the patient. Likewise, a “distal length” of, for example, the catheter includes a length of the catheter intended to be near or in the patient when the catheter is used on the patient. A “distal end” of, for example, the catheter includes an end of the catheter intended to be near or in the patient when the catheter is used on the patient. The distal portion, the distal end portion, or the distal length of the catheter can include the distal end of the catheter; however, the distal portion, the distal end portion, or the distal length of the catheter need not include the distal end of the catheter. That is, unless context suggests otherwise, the distal portion, the distal end portion, or the distal length of the catheter is not a terminal portion or terminal length of the catheter.

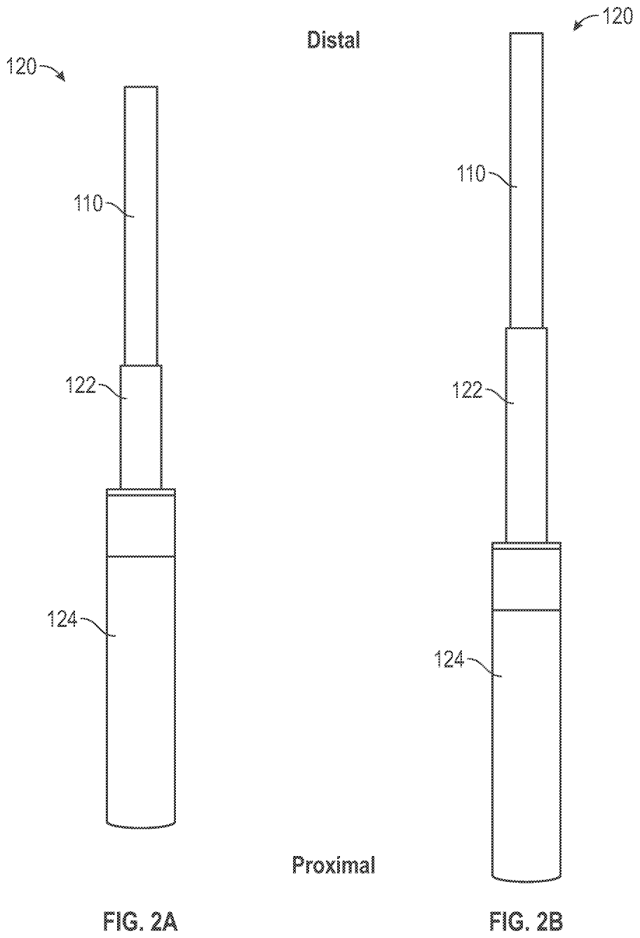

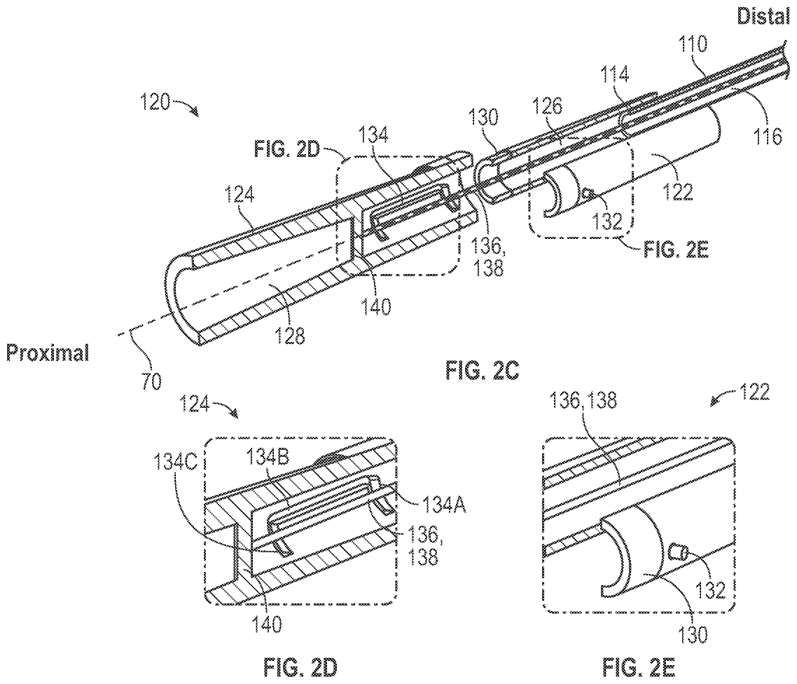

To assist in the description of embodiments described herein, as shown in FIG. 2C, a longitudinal axis extends substantially parallel to an axial length of the catheter. A lateral axis extends normal to the longitudinal axis, and a transverse axis extends normal to both the longitudinal and lateral axes.

Unless defined otherwise, all technical and scientific terms used herein have the same meaning as commonly understood by those of ordinary skill in the art.

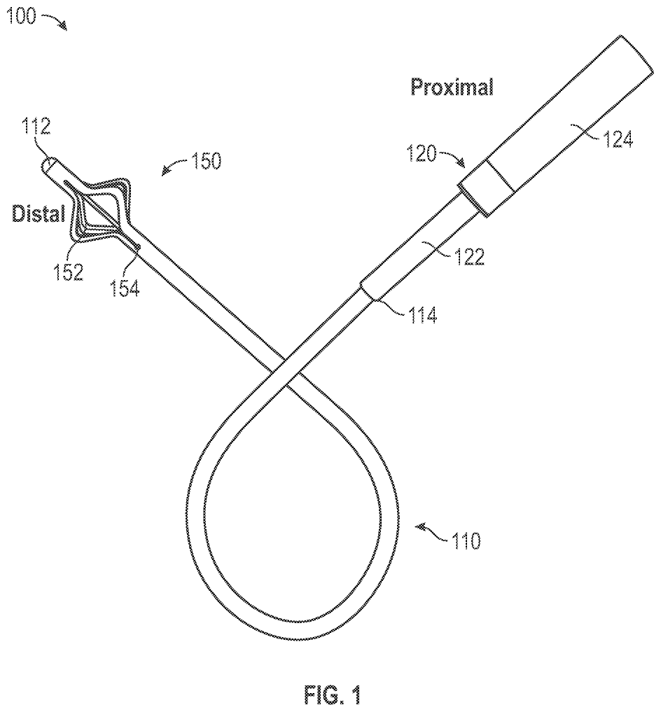

FIG. 1 shows an exemplary bulge-anchor urinary catheter (“catheter”) 100, generally including a catheter body 110, a locking handle (“handle”) 120, and an anchor 150. The catheter body 110 can include a substantially tubular structure, defining one or more lumen 116 and extending longitudinally between a distal tip 112 and a proximal end 114. The catheter body 110 can be formed of a flexible material such as a plastic, polymer, elastomer, rubber, silicone rubber, or the like. The locking handle 120 can be coupled to a proximal end of the catheter body 110 and can be configured to transition the anchor 150 between an insert mode and an anchor mode. Further, the handle 120 can maintain the anchor in one of the insert mode and an anchor mode until repositioned. In an embodiment, the handle 120 can include an indicator, e.g. one or more colored portions, symbols, alphanumeric symbols, or the like, to indicate if the handle is in one of the first configuration or the second configuration. For example, a portion of the male piece 122 can include the indicator that is then covered in the first configuration.

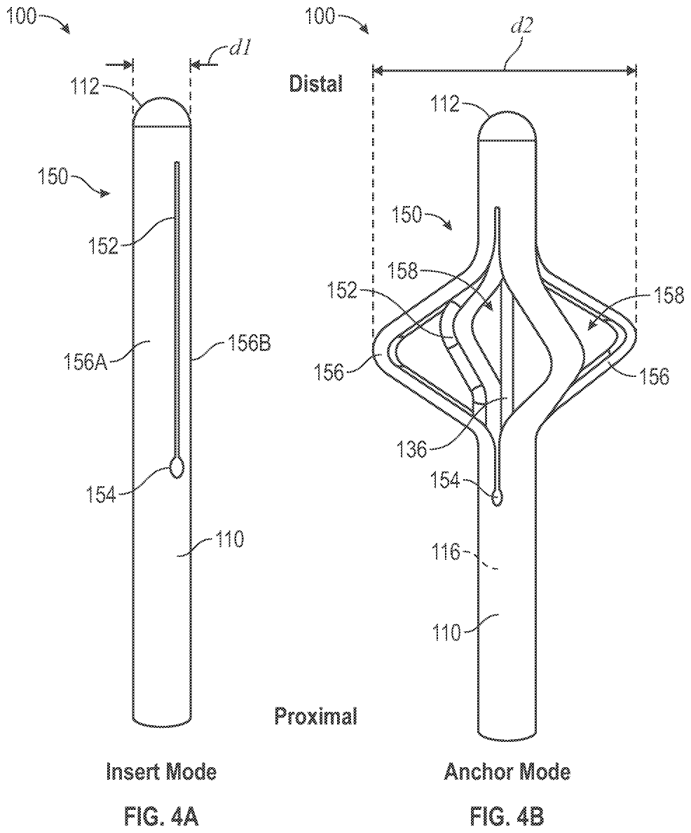

In an embodiment, the anchor 150 can transition between the insert mode (e.g. FIG. 4A) and the anchor mode (e.g. FIG. 4B). In the insert mode the anchor 150 can define a first outer-most diameter (d1). In the anchor mode the anchor 150 can define a second outer-most diameter (d2) that is larger than the first diameter (d1). The first diameter (d1) can be equal to or less than an inner diameter of a urethra. The second diameter (d2) can be greater than an inner diameter of a urethra. In the insert mode the anchor 150 can be configured to pass through the urethra and into the bladder 90. In the anchor mode, the anchor 150 can prevent the distal tip 112 from being withdrawn proximally from the bladder 90.

FIGS. 2A-2E show further details of the locking handle 120, including a male piece, or first handle 122, slidably engaged with a female piece, or second handle 124. FIG. 2A shows the locking handle 120 in a first configuration, for example the insert mode. FIG. 2B shows the locking handle 120 in a second configuration, for example the anchor mode. It will be appreciated, however, that, in an embodiment, the first configuration can be the anchor mode and the second configuration can be insert mode. As shown, the male piece 122 is disposed distally of the female piece 124, however, it will be appreciated other configurations of locking handle 120 are contemplated including the female piece 124 being disposed distally of the male piece 122. These and other configurations of locking handle 120 are contemplated to fall within the scope of the present invention

FIG. 2C shows an exploded view of the handle 120. FIG. 2D shows close up detail of the female piece 124 and FIG. 2E shows close up detail of the male piece 122. The male piece 122 can define a male piece lumen 126 and the female piece 124 can define a female piece lumen 128. A distal end of the male piece 122 can be coupled to a proximal end 114 of the catheter body 110 and the male piece lumen 126 can be in fluid communication with the lumen 116 of the catheter body 110. In an embodiment, the outer diameter of the catheter body 110 can be equal to an inner diameter of a distal end of the male piece lumen 126. The catheter body 110 can engage the male piece 122 in an interference fit, press fit, or snap fit engagement. In an embodiment, the catheter body 110 can be coupled to the male piece 122 using adhesive, bonding, welding, ultrasonic welding, or similar suitable means. In an embodiment, the outer diameter of the male piece 122 can be equal to an inner diameter of a distal end of the female piece lumen 128. As such, the male piece lumen 126 and catheter body lumen 116 can be in fluid communication with the female piece lumen 128.

In an embodiment, the male piece 122 can slidably engage the female piece lumen 128 along a longitudinal axis and can provide a fluid tight seal therebetween. In an embodiment, the handle 120 can further include an O-ring 130 formed of an elastomer, rubber, silicone rubber, or similar suitable material. The O-ring 130 can be coupled to a proximal end of the male piece 122 to ensure a fluid tight seal between the male piece lumen 126 and the female piece lumen 128 while still allowing the male piece 122 and the female piece 124 to slide relative to each other.

The male piece 122 can further include a post 132 extending from an outer surface of the male piece 122 along an axis extending perpendicular to a central longitudinal axis 70. In an embodiment, the handle 120 can include two or more posts 132, for example the handle 120 can include a first post 132 extending from a first side and a second post (not shown) extending from a second side, opposite the first post 132. However, it will be appreciated that other numbers and configurations of posts 132 are also contemplated.

The post 132 can slidably engage a channel 134 disposed on an inner surface of the female piece lumen 128. The channel 134 can substantially define a U-shape, having a distal lateral channel 134A, a longitudinal channel 134B, and a proximal lateral channel 134C. When the post 132 is located in one of the distal lateral channel 134A or the proximal lateral channel 134C, the post 132 can inhibit longitudinal movement of the male piece 122 relative to the female piece 124. When the post 132 is located in the longitudinal channel 134B, the post 132 can slide longitudinally and allow the male piece 122 to slide longitudinally relative to the female piece 124.

In an embodiment, the catheter 100 can further include a string 136 or a push rod 138 extending longitudinally through the catheter lumen 116. A proximal end of the string 136 or push rod 138 can be coupled to the female piece 124, for example at a cross bar 140. To note, while one of the string 136, push rod 138, or cross bar 140 may extend into one or more of the catheter lumen 116, male piece lumen 126, and the female piece lumen 128, these structures provide no impact, or a de minimis impact, on fluid flow therethrough.

In use, with the handle 120 in the first configuration (FIG. 2A), the post 132 can be located within the proximal lateral channel 134C and can prevent the male piece 122 from sliding relative to the female piece 124. To transition the handle 120 from the first position (FIG. 2A) to the second position (FIG. 2B), the clinician can rotate the male piece 122 relative to the female piece 124 about the central longitudinal axis 70 until the post 132 enters the longitudinal channel 134B. The male piece 122 can then slide longitudinally relative to the female piece 124 until the handle 120 is transitioned to the second configuration. The clinician can rotate the male piece 122 relative to the female piece 124 in an opposite direction about the central longitudinal axis 70 until the post 132 enters the distal lateral channel 134A to lock the handle 120 in the second configuration (FIG. 2B). Advantageously, the position of the distal lateral channel 134A and the proximal lateral channel 134C can ensure that the anchor 150 is correctly expanded to the anchor mode and correctly retracted to the insert mode prior to removal. This can prevent over or under expansion of the anchor 150 to ensure correct placement and ensure the anchor 150 is fully retracted to the insert mode before removing the anchor 150 from the bladder 90. In an embodiment, the handle 120 can be biased towards one of the first configuration or the second configuration. In an embodiment, the handle 120 can be bi-stable in both the first configuration and the second configuration.

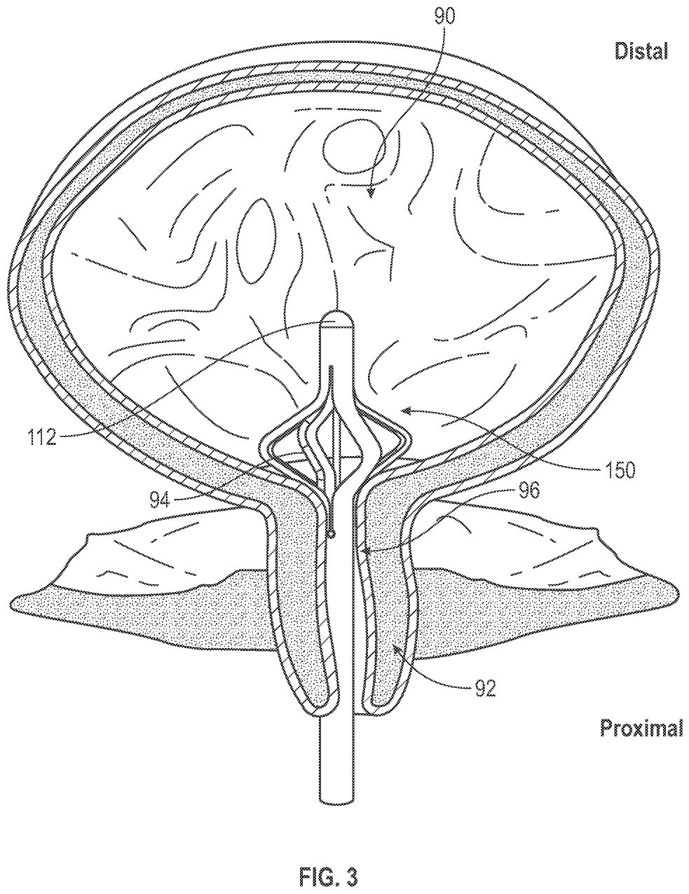

With continued reference to FIG. 1, the catheter can include a malecot-style bulge anchor (“malecot anchor”) 150. Further details of the malecot anchor 150 are shown in FIGS. 3-4B. FIG. 4A shows the malecot anchor 150 in the insert mode. FIG. 4B shows the malecot anchor 150 in the anchor mode. The malecot anchor 150 can include one or more slit valves, “slits” 152, extending longitudinally. A distal end of the slit 152 can extend to a point that is proximal of the distal tip 112 of the catheter body 110. A proximal end of the slit 152 can extend to a point that is distal of an indicator aperture 154. In an embodiment, the slit 152 can communicate with an indicator aperture 154, disposed at a proximal end thereof.

As shown, the malecot anchor 150 can include four slits 152 disposed equidistant about a circumference of the catheter body 110. As such, the slits 152 can define four arms 156, extending longitudinally. It will be appreciated, however, that other numbers and configurations of slits 152 and arms 156 are also contemplated. In an embodiment, the malecot anchor 150 can include a single indicator aperture 154 communicating with one of the slits 152. In an embodiment, the malecot anchor can include a one or more indicator apertures 154, each indicator aperture 154 communicating with one of the one or more slits 152. It will be appreciated, however, that other numbers and configurations of slits 152 and indicator apertures 154 are also contemplated.

The malecot anchor 150 can transition between an insert mode (FIG. 4A) and an anchor mode (FIG. 4B). In the insert mode, the malecot anchor 150 can define a first outer-most diameter (d1). The slit 152 is in a closed position where a first side wall of a first arm 156A contacts a second side wall of a second arm 156B and forming a fluid tight seal therebetween. As such, the slit 152 in the closed position prevents any fluid flow therethrough. In the anchor mode (FIG. 4B), a distal end of the string 136, which is coupled to the distal tip 112 of the catheter body 110 is retracted proximally causing the one or more arms 156 to elastically deform radially outwards and defining a second outer-most diameter (d2). As the arms 156 elastically deform outwards, the slits 152 transition to an open position providing one or more drainage apertures 158 in fluid communication with the catheter lumen 116. To note, the indicator aperture 154 can provide fluid communication with the catheter lumen 116 in both the insert mode and the anchor mode. The drainage aperture 158 can only provide fluid communication with the catheter lumen 116 in the anchor mode.

In use, with the malecot anchor 150 in the insert mode, a clinician can advanced the malecot anchor 150 through the urethra 92 of the patient and into the bladder 90. When the indicator aperture 154 enters the bladder 90 a first fluid flow can immediately enter the catheter lumen 116 and flow proximally to a proximal end of the handle 120. The fluid can then be collected in a suitable container (urine bag, container, toilet, etc.). Optionally, a fluid collection bag can be coupled to the handle 120 can be in fluid communication with the catheter lumen 116. Advantageously, the first fluid flow can be sufficient to indicate to the clinician that the anchor 150 is correctly positioned within the bladder 90, while being sufficiently low enough of a flow that the clinician can cease the flow of fluid 94 as needed. Optionally, the clinician can withdraw the catheter 100 proximally, until the indicator aperture 154 reenters the neck 96 of the bladder 90, to cease all fluid flow as needed.

When the anchor 150 is within the bladder 90, as indicated by the first fluid flow, the clinician can transition the handle 120 from the first configuration to the second configuration and transition the malecot anchor 150 to the anchor mode. In the anchor mode the arms 156 are elastically deformed to define the second diameter (d2) and the slits 152 are opened to provide a second fluid flow through the drainage aperture(s) 158. The second fluid flow can be greater than the first fluid flow, and can drain the fluid 94 from the bladder 90.

The clinician can retract the malecot anchor 150 proximally until the arms 156 engage the neck 96 of the bladder 90 and prevent further proximal movement. As shown in FIG. 3, in such a position the drainage aperture 158 can be disposed at the neck 96 of the bladder 90 and can drain all of the fluid 94 from the bladder 90, preventing any fluid from stagnating. To remove the catheter 100 from the bladder 90, the process can be reversed. In an embodiment, the arms 156 can bias the catheter 100 towards the insert mode.

Advantageously, the indicator aperture 154 can confirm to a clinician when the anchor 150 is within the bladder 90 and can prevent the anchor 150 from being transitioned to the anchor mode prematurely, causing trauma to the urethra 92 or the neck 96 of the bladder 90. Similarly, the indicator aperture 154 can prevent over insertion of the catheter 100 into the bladder 90, where a distal tip 112 impinges on a wall of the bladder 90 causing pain or discomfort to the patient. Advantageously, transitioning the anchor 150 between the insert mode and the anchor mode does not require adding additional volume to the bladder 90 which can cause trauma, pain, discomfort, or bladder spasms to the patient.

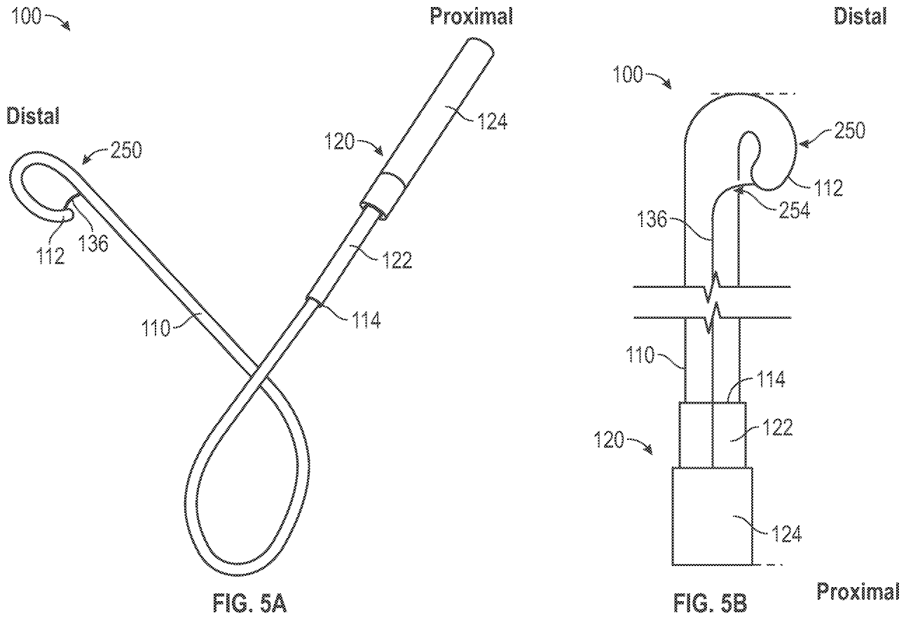

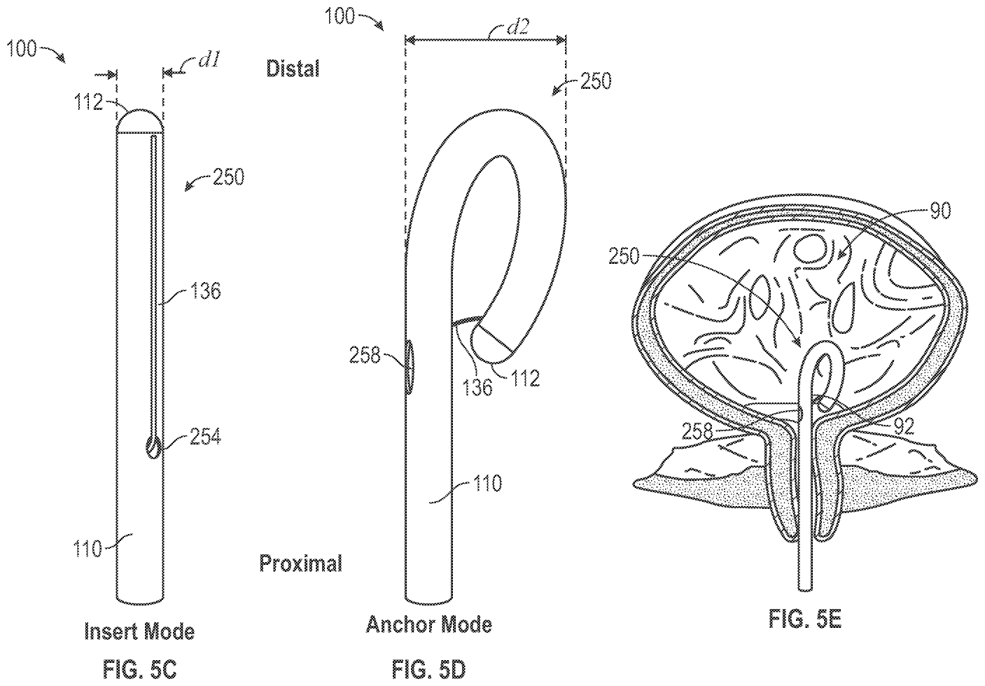

FIGS. 5A-5E show details of a catheter 100 including a loop anchor 250.

FIG. 5A shows a perspective view of a catheter 100 including a handle 120 and the loop anchor 250. FIG. 5B shows a schematic view of the catheter 100 including the handle 120 and the loop anchor 250. FIG. 5C shows the loop anchor 250 in an insert mode. FIG. 5D shows the loop anchor 250 in an anchor mode. FIG. 5E shows the loop anchor 250 in an anchor mode disposed within a bladder 90.

The loop anchor 250 can include an indicator aperture 254 disposed at a first longitudinal position, and a drainage aperture 258 disposed at a second longitudinal position, proximal of the first longitudinal position. The indicator aperture 254 can define a relatively small aperture diameter, and as such can provide a first fluid flow. The drainage aperture 258 can define a relatively large aperture diameter, and as such can provide a second fluid flow, greater than the first fluid flow. The loop anchor 250 can further include a string 136 extending through the catheter lumen 116 to the indicator aperture 254 and can extend through the indicator aperture 254 and over an outer surface of the loop anchor 250. A distal tip of the string 136 can be coupled to an outer surface of the distal tip 112 of the catheter body 110.

In use, the loop anchor 250 in the insert mode can be urged through the urethra 92 until the loop anchor 250 enters the bladder 90. When the indicator aperture 254 enters the bladder 90, a first fluid flow can flow through the catheter body lumen 116 to indicate that the entire loop anchor 250 is within the bladder 90. The clinician can then actuate the handle 120, as described herein, to retract the string 136 and transition the loop anchor 250 to the anchor mode. In the anchor mode, the distal tip 112 can be bent towards the indicator aperture 254 to create a loop having a second outer-most diameter (d2). The loop anchor 250 in the anchor mode can prevent the distal tip 112 from being withdrawn proximally from the bladder 90.

Once the loop anchor 250 has been secured in the anchor mode, the clinician can adjust the catheter body 110 proximally or distally until the drainage aperture 258 is positioned at the neck 96 of the bladder 90 to provide a second fluid flow, greater than the first fluid flow, and drain all of the fluid 94 from the bladder 90. In an embodiment, the loop anchor 250 can be advanced into the bladder 90 and transitioned to the anchor mode. The catheter 100 can then be withdrawn proximally until a distal tip 112 impinges against a wall of the bladder 90. The longitudinal position of the drainage aperture 258 can be such that when the distal tip 112 impinges against the wall of the bladder 90, the drainage aperture 258 is aligned with the neck 96 of the bladder and drains all of the fluid from the bladder 90.

To note, a distal portion of the catheter body 110 can be biased towards the insert mode, and can be elastically deformed to the anchor mode. As such, to transition the loop anchor 250 from the anchor mode to the insert mode, the clinician can transition the handle 120 to from the second configuration to the first configuration to allow the distal portion of the catheter 110 to resume a linear configuration.

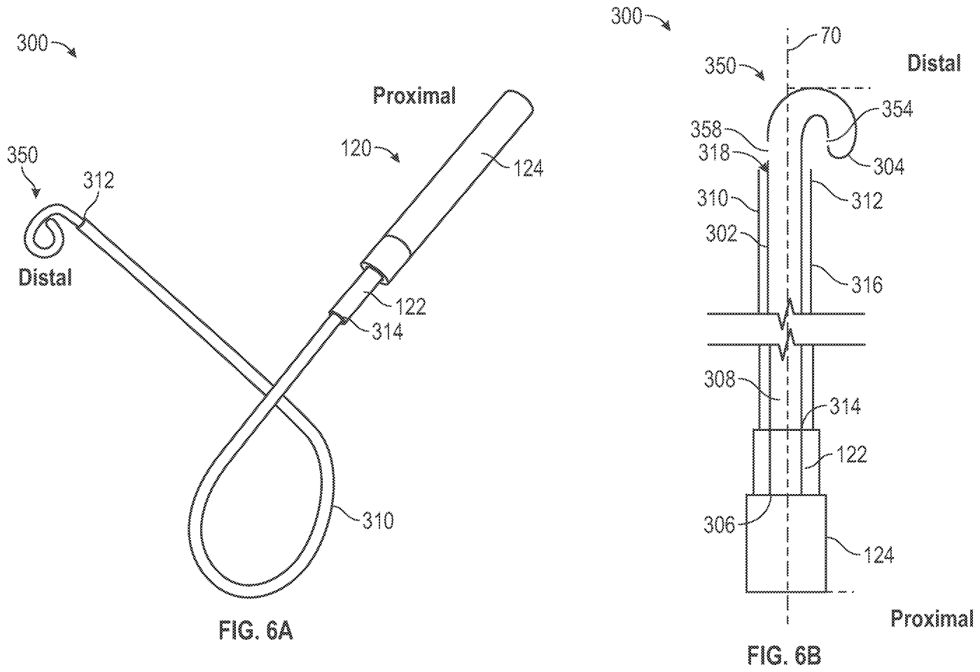

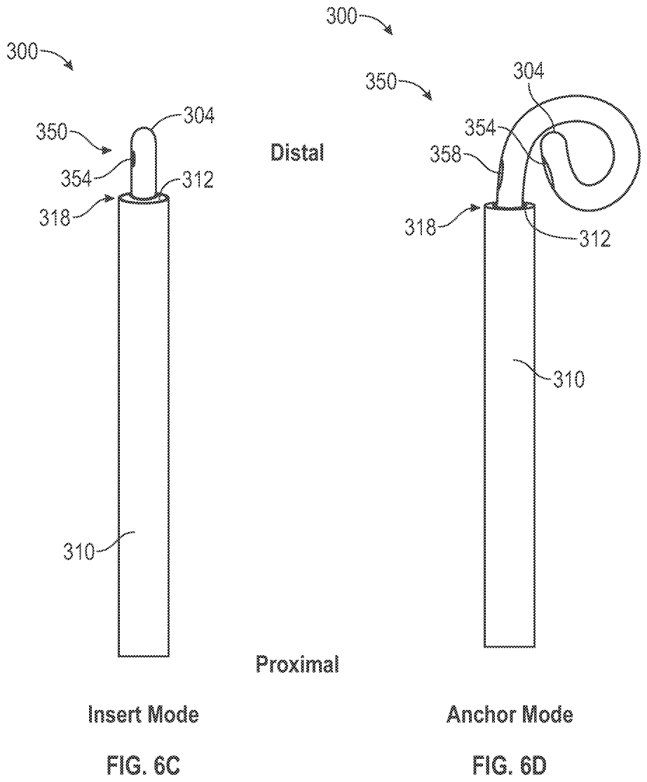

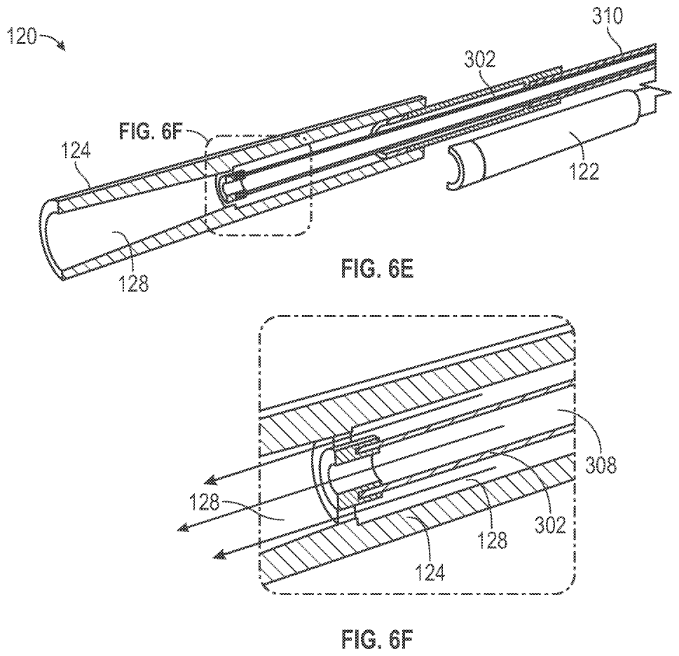

FIGS. 6A-6F show an embodiment of a catheter 300 including a locking handle 120 and a J-anchor 350. The catheter 300 can include an outer catheter body 310 and an inner catheter body 302 each extending co-axially along a central longitudinal axis 70. The outer catheter body 310 can define a lumen 316 and extend longitudinally between a proximal end 314 and a distal tip 312. The proximal end 314 of the outer catheter 310 can be coupled to a male piece 122 of a handle 120, as described herein. A distal tip 312 of the outer catheter 310 can define a distal opening 318. The inner catheter body 302 can also extend longitudinally between a proximal end 306 and a distal tip 304. A proximal end 306 of the inner catheter body 302 can be coupled to the female piece 124. The inner catheter body 306 can define an inner catheter body lumen 308 that is in fluid communication with the female piece lumen 128.

An outer diameter of the inner catheter body 310 can be equal to or slightly less than an inner diameter of the outer catheter body lumen 316. As such, the inner catheter body 302 can slidably engage the outer catheter body 310, along a longitudinal axis 70, while maintaining a substantially fluid-tight seal therebetween. In an embodiment, as shown in FIGS. 6E-6F, any fluid that enters the outer catheter body lumen 316, along an outer surface of the inner catheter body 302, can drain proximally and enter the male piece lumen 126 the female piece lumen 128, and be collected in a suitable container, as described herein.

In an embodiment, a distal end of the inner catheter body 302 can include a J-anchor 350. The J-anchor 350 can include a portion of the inner catheter body 302 that defines a non-linear, curved, or “pig-tail” shape. In an embodiment, the non-liner shape of the J-anchor 350 can be maintained by varying wall thicknesses between a first side and a second side of a distal portion of the inner catheter body 302. The J-anchor 350 can be elastically deformed from the non-linear shape to a different non-linear shape or to a linear shape. Further, the J-anchor 350 can include an indicator aperture 354 and a drainage aperture 358, each extending through a wall of the inner catheter 302 and communicating with the inner catheter lumen 308. The indicator aperture 354 can define a first aperture diameter, and the drainage aperture 358 can define a second aperture diameter. The first aperture diameter can be less than the second aperture diameter. The indicator aperture 354 can be disposed at a distal tip 304 of the inner catheter body 302. The drainage aperture 358 can be disposed at a proximal end of the J-anchor 350.

In use, as shown in FIGS. 6C-6D, in the insert mode, the J-anchor 350 can be withdrawn proximally into the outer catheter 310. The inner catheter body 302 can be formed of a more flexible material relative to the outer catheter body 310. As such, the J-anchor 350 can be elastically deformed to a substantially linear configuration within the outer catheter body 310. A distal tip 304 of the inner catheter body 302, including the indicator aperture 354 can be positioned adjacent to, or proximal of the distal opening 318 of the outer catheter body 310.

The catheter 300 can be advanced through the urethra 92 until the indicator aperture 354 enters the bladder 90. A first fluid flow can flow proximally through the inner catheter lumen 308 to the female piece lumen 128. The first fluid flow can indicate to the clinician that the distal tip 112 of the outer catheter body 310 is within the bladder 90 and the J-anchor 350 is safe to deploy. The clinician can then transition the handle 120 from the second configuration (FIG. 2B) to the first configuration (FIG. 2A) to extend the J-anchor 350 distally of the distal end opening 318 of the outer catheter body 310. As such, the J-anchor 350 can resume the non-linear resting position of the anchor mode (FIG. 6D). In the anchor mode the drainage aperture 358 is extended distally of the distal opening 318 and can provide a second fluid flow, greater than the first fluid flow. Further the drainage aperture 358 can be positioned adjacent a neck 96 of the bladder 90 to ensure all the fluid is drained from the bladder 90.

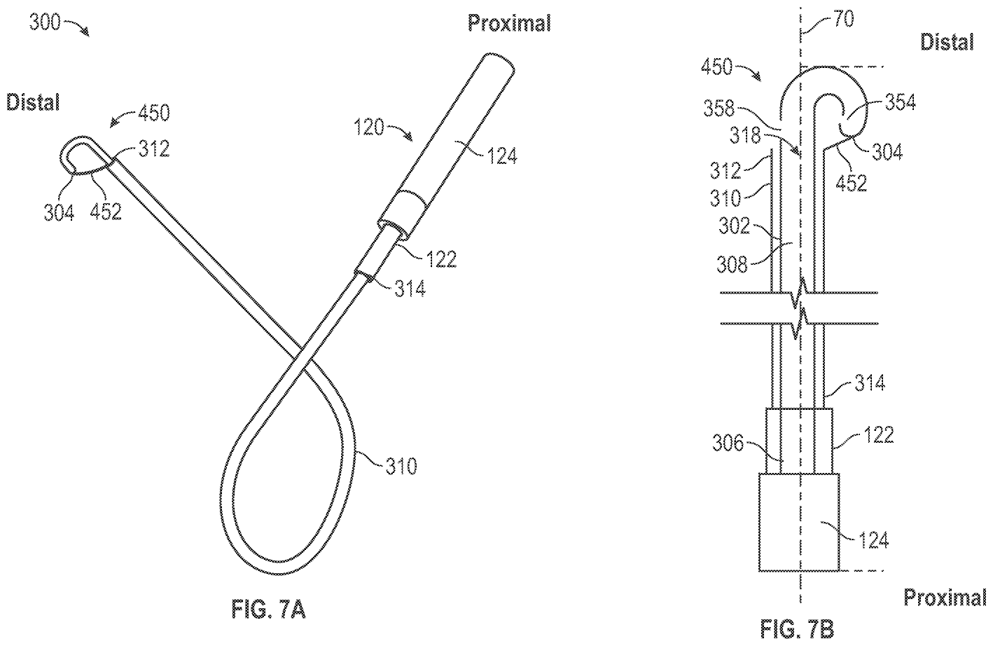

In an embodiment, as shown in FIGS. 7A-7D, the catheter 300 can include a tethered J-anchor 450. The catheter 300 can include an inner catheter body 302 co-axially aligned with, and slidable engaged with, an outer catheter 310. The proximal end 314 of the outer catheter body 310 can be coupled with the male piece 122 and the proximal end 306 of the inner catheter body 302 can be coupled with female piece 124 of the handle 120, as described herein. The tethered J-anchor 450 can define a substantially linear configuration and can be elastically deformed to a non-linear shape in the anchor mode. The tethered J-anchor 450 can further include a tether 452 extending from a distal tip 312 of the outer catheter body 310 and the distal tip 304 of the inner catheter body 302.

In use, the catheter 300 can be advanced through the urethra 92 until the indicator aperture 354 enters the bladder 90 and provides a first fluid flow, as described herein. The clinician can transition the handle 120 from the second configuration (FIG. 2B) to the first configuration (FIG. 2A). The inner catheter body 302 can be urged distally relative to the outer catheter body 310. The tether 452 prevents the distal tip 304 of the inner catheter body 302 from advancing in a linear direction and instead causes the tethered J-anchor 450 to elastically deform to a non-linear, curved, or “pig-tail” shape, as shown in FIGS. 7A, 7B. With the handle 120 in the first configuration, the drainage aperture 358 can be advanced distally of the distal opening 318 and provide a second fluid flow, greater than the first fluid flow. The tethered J-anchor 450 in the anchor mode can prevent the distal tip 312 of the outer catheter body 310 from being withdrawn proximally from the bladder 90.

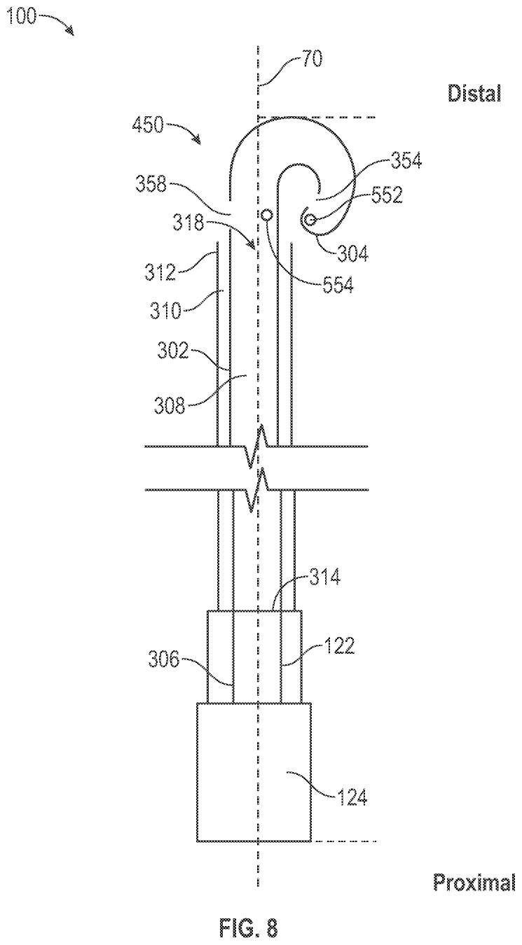

In an embodiment, as shown in FIG. 8, the catheter 300 can include a magnetic J-anchor 550. The magnetic J-anchor 550 can include a first magnetic element 552 disposed in a wall of the inner catheter body 302, adjacent the distal tip 304. The magnetic J-anchor 550 can include a second magnetic element 554 disposed in a wall of the inner catheter body 302 adjacent the drainage aperture 358. As used herein, a magnetic element can include a permanent magnet, electro-magnet, a magnetized ferrous material, or a ferrous material.

In use, the catheter 300 in the insert mode can include the magnetic J-anchor 550 in a linear configuration disposed within a distal portion of the outer catheter 310. When the indicator aperture 354 enters the bladder 90, a first fluid flow can indicate that the distal tip 312 of the outer catheter 310 has entered the bladder 90. The clinician can then transition the handle 120 from the second configuration (FIG. 2B) to the first configuration (FIG. 2A) to advance the magnetic J-anchor 550, including the second magnetic element 554, distally of the distal opening 318. As such, the first magnetic element 552 can be attracted to the second magnetic element 554 and can elastically deform the magnetic J-anchor 550 to the anchor mode and prevent the catheter 300 from being withdrawn proximally. In the anchor mode, the drainage aperture 358 can be disposed distally of the distal opening 318 to provide a second fluid flow through the inner catheter lumen 308 to drain the bladder 90 of fluid, as described herein.

To retract the catheter 300 from the bladder 90, the clinician can transition the handle 120 from the first configuration (FIG. 2A) to the second configuration (FIG. 2B). The handle 120 can retract the inner catheter 302 into the outer catheter 310, urging a wall of the outer catheter 310 between the first magnetic element 552 and the second magnetic element 554. Separating the first magnetic element 552 and the second magnetic element 554 allows the magnetic J-anchor 550 to return to the linear configuration of the insert mode and be withdrawn into the outer catheter lumen 316.

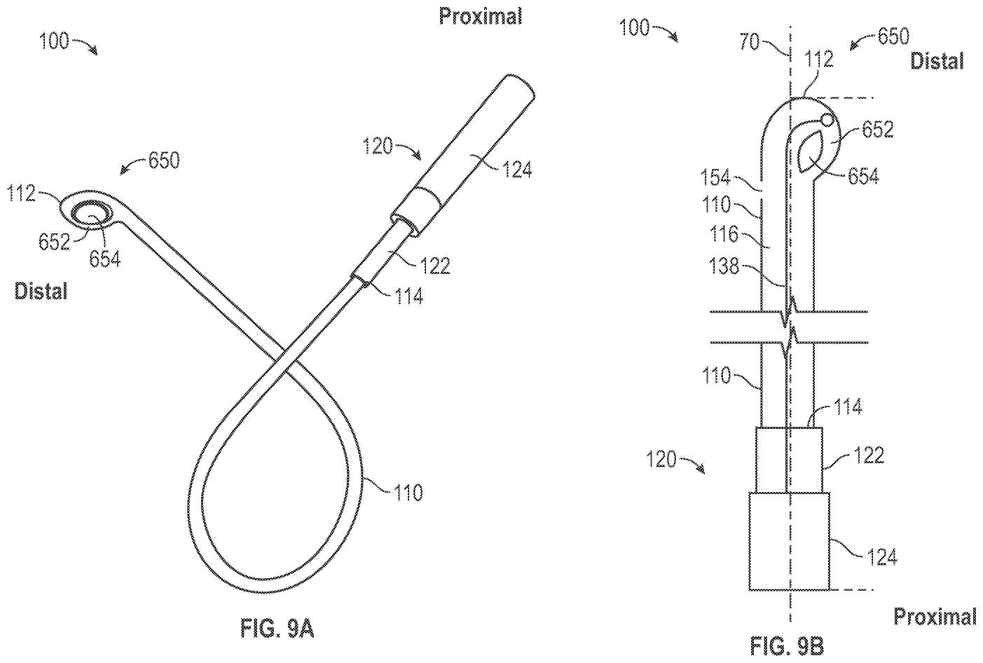

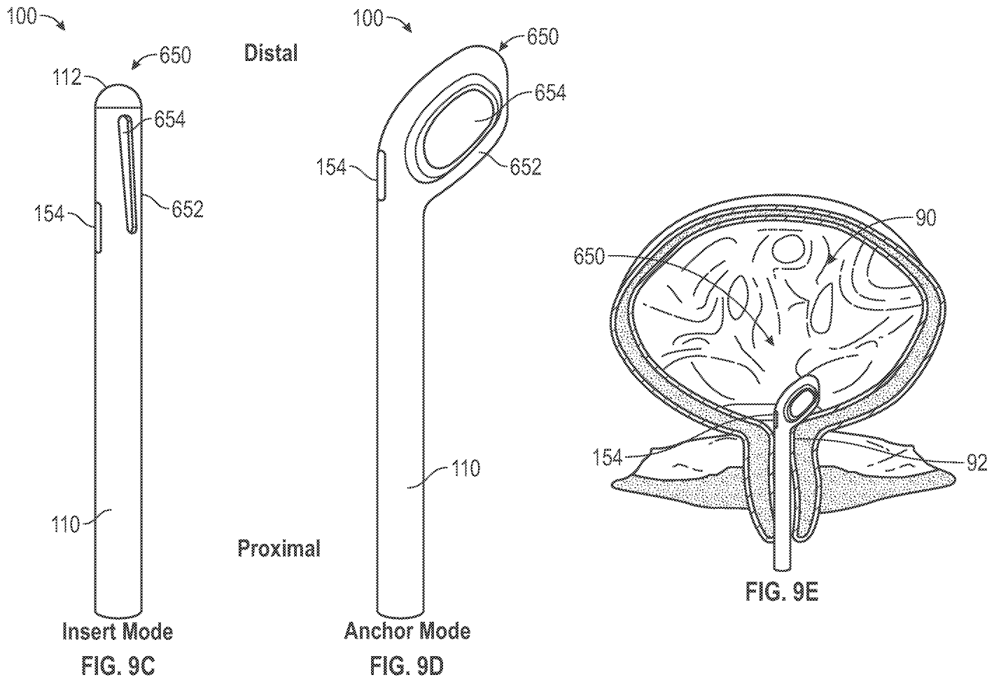

FIGS. 9A-9E show a catheter 100 including a push-rod anchor 650 disposed at a distal end thereof. The catheter 100 can include a lumen 116 extending from a closed distal tip 112 to the handle 120, as described herein. The push-rod anchor 650 can include an indicator aperture 154 disposed at a proximal end of the push-rod anchor 650 and can include a tether 652 extending from a distal tip 112 of the catheter body 110 to a proximal end of the push-rod anchor 650. In an embodiment, the tether 652 can be formed integrally with the catheter body 110, extending over an outer surface of the catheter body 110.

Worded differently, a distal portion of the catheter body 110 defining the push-rod anchor 650 can include a tether aperture 654 extending between a first side of the catheter body 110 to a second side of the catheter body 110, opposite the first side. As such, the tether aperture 654 can define the tether 652. In an embodiment, the tether 652 can be formed as a solid structure and formed of the same material as the catheter body 110. In an embodiment, the tether 652 can be formed as a hollow structure and is in fluid communication with the catheter lumen 116. In an embodiment, the tether 652 can be formed as a separate structure from that of the catheter body 110, and can be coupled to the catheter body 110 using adhesive, bonding, welding, or similar suitable means. The tether 652 can be formed of the same material or a different material from that of the catheter body 110.

In an embodiment, the catheter 100 can include a push-rod 138 extending through the catheter lumen 116. A proximal end of the push-rod 138 can be coupled with the female piece 124 of the handle 120, for example the cross-bar 140. A distal tip of the push rod 138 can extend through the catheter lumen 116 to a distal tip 112 of the catheter body 110. In an embodiment, a distal end of the push rod 138 can include an atraumatic tip, ball tip, or similar slug coupled to the distal tip of the push rod 138 and configured to prevent trauma to the catheter body 110. In an embodiment, the push rod 138 can be formed of a plastic, polymer, metal, alloy, composite, or similar suitable material. In an embodiment, the push-rod anchor 650 can transition between the insert mode (FIG. 9C) and the anchor mode (FIG. 9D). The push-rod anchor 650 can be biased towards the insert mode and can be elastically deformed to the anchor mode.

In use, with the push-rod anchor 650 in the insert mode, the clinician can advance the distal tip 112 of the catheter 110 through the urethra 92 into the bladder 90. The indicator aperture 154 disposed at the proximal end of the push-rod anchor 650 can enter the bladder 90 and provide one of a first fluid flow or a second fluid flow indicating that the push-rod 650 is fully disposed within the bladder 90. The clinician can then transition the handle 120 from the second configuration (FIG. 2B) to the first configuration (FIG. 2A) to advance the push rod 138 distally relative to the catheter body 110. As such, the distal tip of the push rod 138 can impinge on a distal tip 112 of the catheter 110 and elastically deform the push-rod anchor 650 to the anchor mode, and prevent the distal tip 112 from being withdrawn proximally from the bladder 90. In an embodiment, the indicator aperture 154 in the insert mode can define a first aperture diameter and can provide a first fluid flow. The push-rod anchor 650 in the anchor mode can elastically deform the indicator aperture 154 from the first aperture diameter to the second aperture diameter and provide a second fluid flow, greater than the first fluid flow.

While some particular embodiments have been disclosed herein, and while the particular embodiments have been disclosed in some detail, it is not the intention for the particular embodiments to limit the scope of the concepts provided herein. Additional adaptations and/or modifications can appear to those of ordinary skill in the art, and, in broader aspects, these adaptations and/or modifications are encompassed as well. Accordingly, departures may be made from the particular embodiments disclosed herein without departing from the scope of the concepts provided herein.

1. An indwelling urinary catheter, comprising:
a catheter body defining a lumen; and
an anchor disposed at a distal end of the catheter body and transitionable between an insert mode and an anchor mode, the anchor comprising:
a first slit valve and a second slit valve each extending longitudinally and defining a first arm and a second arm;
an indicator aperture disposed proximally of one or both of the first slit valve and the second slit valve, and configured to provide a first fluid flow to the lumen in both the insert mode and the anchor mode; and
wherein the first slit valve and the second slit valve are each configured to define a drainage aperture in the anchor mode to allow a second fluid flow into the lumen and to prevent the second fluid flow into the lumen in the insert mode.

2. The indwelling urinary catheter according to claim 1, wherein the first fluid flow is less than the second fluid flow.

3. The indwelling urinary catheter according to claim 1, wherein the indicator aperture communicates with one of the first slit valve or the second slit valve.

4. The indwelling urinary catheter according to claim 1, wherein one or both of the indicator aperture and the drainage aperture extends through a side wall of the catheter body to communicate with the lumen.

5. The indwelling urinary catheter according to claim 1, wherein the first arm and the second arm define a first outer diameter in the insert mode and a second outer diameter in the anchor mode, the first diameter being less than the second diameter.

6. The indwelling urinary catheter according to claim 1, further including a locking handle coupled to a proximal end of the catheter body, and a string extending through the lumen, a proximal end of the string is coupled to a female piece of the locking handle, and a distal end of the string is coupled to a distal tip of the catheter body, a proximal end of the catheter body is coupled a male piece of the locking handle, the female piece slidably engaged with the male piece to transition the anchor between the insert mode and the anchor mode.

7. The indwelling urinary catheter according to claim 6, wherein the female piece is configured to slide proximally relative to the male piece to transition the anchor to the anchor mode.

8. The indwelling urinary catheter according to claim 6, wherein the female piece includes a channel having a distal lateral channel communicating with a longitudinal channel which communicates with a proximal lateral channel, the male piece includes a post extending from an outer surface thereof, the post slidably engaged with the distal lateral channel in the insert mode, the proximal lateral channel in the anchor mode, and the longitudinal channel when transitioning therebetween.

9. A urinary catheter, comprising:
an outer catheter including a distal opening, and a first handle coupled to a proximal end thereof, and
an inner catheter arranged co-axially and slidably engaged with the outer catheter, the inner catheter comprising:
an anchor disposed distally and transitionable between an insert mode and an anchor mode;
an indicator aperture disposed at a distal tip of the inner catheter;
a drainage aperture disposed proximally of the anchor; and
a second handle coupled to a proximal end thereof.

10. The urinary catheter according to claim 9, wherein the anchor is configured to be advanced distally of the distal opening to transition to the anchor mode, and withdrawn proximally of the distal opening to transition to the insert mode.

11. The urinary catheter according to claim 9, wherein the first handle is co-axially arranged and slidably engaged with the second handle along a longitudinal axis between a first configuration and a second configuration to transition the anchor between the insert mode and the anchor mode.

12. The urinary catheter according to claim 11, wherein the first handle includes a post extending from an outer surface thereof and the second handle includes a channel having a distal lateral channel communicating with a longitudinal channel which communicates with a proximal lateral channel, the post slidably engaged with the proximal lateral channel in the insert mode, slidably engaged with the distal lateral channel in the anchor mode, and slidably engaged with the longitudinal channel when transitioning between the insert mode and anchor mode.

13. The urinary catheter according to claim 9, wherein the anchor is biased towards one of a J-shape, or a pigtail shape, in the anchor mode and elastically deformed to a linear shape in the insert mode when the anchor is withdrawn proximally into a distal portion of the outer catheter.

14. The urinary catheter according to claim 9, wherein the anchor is biased towards a linear shape and further includes a tether extending between a distal tip of the inner catheter and a distal tip of the outer catheter, the tether configured to elastically deform the anchor to a non-linear configuration when the anchor is advanced distally of the distal opening.

15. The urinary catheter according to claim 9, wherein the anchor is biased towards a linear shape and further includes first magnetic element disposed in a distal tip of the inner catheter and a second magnetic element disposed in a wall of the inner catheter, proximally of the anchor, the first magnetic element configured to be attracted to the second magnetic element when the second magnetic element is advanced distally of the distal opening and elastically deform the anchor to a non-linear configuration.

16. An indwelling urinary catheter, comprising:
a catheter body defining a lumen;
an anchor structure disposed at a distal end of the catheter body and including a tether integrally formed therewith extending from a distal tip of the catheter body to a proximal end of the anchor structure;
an aperture extending through a wall of the catheter body and communicating with the lumen, the aperture disposed proximally of the anchor structure; and
a push rod extending through the lumen and configured to be urged distally to elastically deform the anchor structure from an insert mode defining a first diameter, to an anchor mode defining a second diameter, larger than the first diameter.

17. The indwelling urinary catheter according to claim 16, wherein the aperture in the insert mode defines a first aperture diameter to provide a first fluid flow, and wherein the aperture in the anchor mode defines a second aperture diameter, larger than the first aperture diameter to provide a second fluid flow, greater than the first fluid flow.

18. A method of draining a fluid from a bladder of a patient, comprising:
advancing a distal tip of a catheter through a urethra into the bladder, the catheter including an anchor, an indicator aperture, and a drainage aperture;
advancing the indicator aperture into the bladder to provide a first fluid flow and indicating the anchor is correctly positioned for transitioning from an insert mode to an anchor mode;
transitioning a locking handle between a first configuration and a second configuration;
transitioning the anchor from the insert mode to the anchor mode; and
providing fluid communication between the drainage aperture and the bladder to provide a second fluid flow, different from the first fluid flow.

19. The method according to claim 18, wherein the drainage aperture includes a slit valve disposed proximally of the indicator aperture and transitions from a closed position in the insert mode which prevents the second fluid flow, to an open position in the anchor mode which provides the second fluid flow.

20. The method according to claim 18, wherein the catheter includes an outer catheter body and an inner catheter body, slidably engaged and co-axially aligned therewith, and wherein transitioning the anchor to the anchor mode includes advancing the anchor and the drainage aperture distally of a distal tip of the outer catheter body.

21. The method according to claim 20, further including a tether extending between a distal tip of the inner catheter body and a distal tip of the outer catheter body and elastically deforming a distal portion of the inner catheter body to a non-linear configuration.

22. The method according to claim 20, further including a first magnetic element disposed in a tip of the inner catheter body, and a second magnetic element disposed in a wall of the inner catheter body proximally of the anchor, and wherein transitioning the anchor to the anchor mode includes advancing the first magnetic element and the second magnetic element distally of the distal tip of the outer catheter body to allow the first magnetic element to couple with the second magnetic element elastically deforming a distal portion of the inner catheter body to a non-linear configuration.

23. The method according to claim 18, wherein transitioning the anchor to an anchor mode further includes advancing a push rod distally through a lumen of the catheter to elastically deform the anchor, the anchor including a tether formed integrally therewith and extending from a distal tip of the catheter to a proximal end of the anchor.

24. An indwelling urinary catheter, comprising:
a catheter body defining a lumen;
a locking handle coupled to the proximal end of the catheter body; and
an anchor disposed at a distal end of the catheter body and transitionable between an insert mode and an anchor mode, the anchor comprising:
an indicator aperture disposed at a first longitudinal position;
a drainage aperture disposed at a second longitudinal position, proximal of the first longitudinal position; and
a string extending from a distal tip of the catheter body over an outer surface of the anchor, through the indicator aperture and through the lumen of the catheter body, a proximal end of the string coupled to the handle.
